(19) United States
(12) Patent Application Publication
(10) Pub. No.: US 2016/0090619 A1
(43) Pub. Date: Mar. 31, 2016

(54) NUCLEIC ACID PURIFICATION DEVICE

(71) Applicant: Seiko Epson Corporation, Tokyo (JP)

(72) Inventor: Toshiro MURAYAMA, Fujimi (JP)

(21) Appl. No.: 14/867,443

(22) Filed: Sep. 28, 2015

(30) Foreign Application Priority Data
Sep. 30, 2014 (JP) 2014-199563

(51) Int. Cl. C12Q 1/68

(52) U.S. Cl. CPC C12Q 1/6806

(57) ABSTRACT

In a nucleic acid purification device, a washing container and an elution container are bonded to each other to form a channel for moving a nucleic acid, the washing container includes an outer peripheral wall which accommodates a connection portion of the first channel and a second channel, the elution container includes a plurality of flanges in the periphery of the second channel in contact with an inner wall of the outer peripheral wall, the plurality of flanges are arranged in a portion which is to be inserted into the inside of the outer peripheral wall of the elution container, and one space which is partitioned by two flanges adjacent to each other among the plurality of flanges and the outer peripheral wall communicates with another space adjacent to the one space in a state of being divided by one of the two flanges adjacent to each other.

### BACKGROUND

1. Technical Field

The present invention relates to a nucleic acid purification device.

2. Related Art

In the field of biochemistry, a technology of a polymerase chain reaction (PCR) has been established. In recent years, precision of amplification or detection sensitivity using the PCR method is improved so that an extremely small amount of specimen (DNA or the like) is amplified and detection and analysis can be performed. The PCR is a method of amplifying a target nucleic acid by applying a thermal cycle to a solution (reaction solution) containing a nucleic acid (target nucleic acid) which is a target of amplification and a reagent. As the method of applying the thermal cycle of the PCR, a method of applying a thermal cycle at a temperature in two stages or three stages is normally used.

Meanwhile, currently, it is the mainstream to use a simple test kit such as an immunochromatograph kit for diagnosis of infectious diseases such as influenza in the field of medical care. However, in such a simple test, the precision is insufficient in some cases and thus it is desired to apply the PCR which can be expected to have high test precision to the diagnosis of infectious diseases.

In recent years, a device that performs purification of a nucleic acid by alternately laminating an aqueous liquid layer and a water-insoluble gel layer in a capillary and allowing magnetic particles to which a nucleic acid is attached to pass through has been suggested as a device using the PCR method or the like (see International Publication No. 2012/086243). However, when such a device is stored for a long period of time, components of the aqueous liquid layer are gradually diffused through the gel layer and one aqueous liquid layer is contaminated by components of another aqueous liquid layer in some cases.

### SUMMARY

An advantage of some aspects of the invention is to provide a nucleic acid purification device that prevents one aqueous liquid layer from being contaminated by components of another aqueous liquid layer even when the device is stored for a long period of time.

### Application Example 1

A nucleic acid purification device according to this application example includes: a washing container in which a washing solution and a fluid which is not mixed with the washing solution are sealed by and stored in a first channel; and an elution container in which an eluate and a fluid which is not mixed with the eluate are sealed by and stored in a second channel, the washing container and the elution container being bonded to each other to form a channel for moving a nucleic acid, in which the washing solution is a liquid which washes a nucleic acid-binding solid phase carrier to which the nucleic acid is adsorbed, the eluate is a liquid which separates the nucleic acid from the nucleic acid-binding solid phase carrier, the washing container includes an outer peripheral wall which is arranged by being spaced apart from the first channel and accommodates a connection portion of the first channel and the second channel, the elution container is arranged in the periphery of the second channel and includes a plurality of flanges in contact with an inner wall of the outer peripheral wall, the plurality of flanges are arranged in a portion which is to be inserted into the inside of the outer peripheral wall of the elution container, and one space which is partitioned by two flanges adjacent to each other among the plurality of flanges and the outer peripheral wall communicates with another space adjacent to the one space in a state of being divided by one of the two flanges adjacent to each other.

In the purification device according to this application example, since the washing container and the elution container respectively seal and store contents until the washing container and the elution container are bonded to each other, it is possible to prevent the eluate from being contaminated by the washing solution. In addition, in the purification device according to the application example, since the mixture of the washing solution with the eluate is prevented by the fluids which are not mixed with the washing solution and the eluate even after the washing container and the elution container are bonded to each other, it is possible to prevent the eluate from being contaminated by the washing solution by promptly using the eluate after assembly. Further, in the purification device according to the application example, leakage of the fluid in the washing container or the fluid in the elution container to the outside of the nucleic acid purification device can be prevented while the air (atmosphere) in the outer peripheral wall escapes to the outside when the washing container and the elution container are bonded to each other (when the washing container is inserted into the elution container). Moreover, in the nucleic acid purification device according to the application example, the plurality of flanges can function as a guide for inserting the washing container into the elution container.

### Application Example 2

In the nucleic acid purification device according to the application example, the elution container may be arranged in the periphery of the second channel and include a seal flange in contact with the inner wall of the outer peripheral wall, the plurality of flanges may be arranged more on the connection portion side than the seal flange, and the seal flange may seal the inner wall of the outer peripheral wall.

In the nucleic acid purification device according to this application example, when the washing container and the elution container are bonded to each other, leakage of the fluid in the washing container or the fluid in the elution container to the outside of the nucleic acid purification device can be more reliably prevented.

### Application Example 3

In the nucleic acid purification device according to the application example, the plurality of flanges may be provided with a notched portion, and the one space may communicate with another space by passing through the notched portion.

In the nucleic acid purification device according to this application example, when the washing container and the elution container are bonded to each other, the air in the outer peripheral wall can escape to the outside of the nucleic acid purification device by passing through the notched portion.

### Application Example 4

In the nucleic acid purification device according to the application example, an outer peripheral portion of the plurality of flanges may be in contact with the inner wall of the outer peripheral wall, excluding the notched portion.

In the nucleic acid purification device according to this application example, the plurality of flanges can more reliably function as a guide for inserting the washing container to the elution container.

### Application Example 5

A nucleic acid purification device according to this application example includes: a washing container which seals and stores a washing solution and a fluid which is not mixed with the washing solution; and an elution container which seals and stores an eluate and a fluid which is not mixed with the eluate, the washing container and the elution container being bonded to each other to form a channel for moving a nucleic acid, in which the washing solution is a liquid which washes a nucleic acid-binding solid phase carrier to which the nucleic acid is adsorbed, the eluate is a liquid which separates the nucleic acid from the nucleic acid-binding solid phase carrier, and a connection portion of the washing container and the elution container is provided with a plurality of annular spaces communicating with each other.

In the purification device according to this application example, since the washing container and the elution container respectively seal and store contents until the washing container and the elution container are bonded to each other, it is possible to prevent the eluate from being contaminated by the washing solution. In addition, in the purification device according to the application example, since the mixture of the washing solution with the eluate is prevented by the fluids which are not mixed with the washing solution and the eluate even after the washing container and the elution container are bonded to each other, it is possible to prevent the eluate from being contaminated by the washing solution by promptly using the eluate after assembly. Further, in the purification device according to the application example, leakage of the fluid in the washing container or the fluid in the elution container to the outside of the nucleic acid purification device can be prevented while the air (atmosphere) in the outer peripheral wall escapes to the outside when the washing container and the elution container are bonded to each other (when the washing container is inserted into the elution container).

### Application Example 6

A nucleic acid purification device according to this application example includes: a first container in which a first liquid and a fluid which is not mixed with the first liquid are sealed by and stored in a first channel; and a second container in which a second liquid and a fluid which is not mixed with the second liquid are sealed by and stored in a second channel, the first container and the second container being bonded to each other to form a channel for moving a nucleic acid, in which the first container includes an outer peripheral wall which is arranged in a state of being spaced apart from the first channel and capable of accommodating a connection portion of the first channel and the second channel, the second container is arranged in the periphery of the second channel and includes a plurality of flanges in contact with an inner wall of the outer peripheral wall, the plurality of flanges are arranged in a portion which is to be inserted to the inside of the outer peripheral wall of the elution container, and one space which is partitioned by two flanges adjacent to each other among the plurality of flanges and the outer peripheral wall communicates with another space adjacent to the one space in a state of being divided by one of the two flanges adjacent to each other.

In the nucleic acid purification device according to this application example, it is possible to prevent one aqueous liquid layer from being contaminated by components of another aqueous liquid layer even when the device is stored for a long period of time.

### BRIEF DESCRIPTION OF THE DRAWINGS

The invention will be described with reference to the accompanying drawings, wherein like numbers reference like elements.

FIG. 1 is a front view illustrating a container assembly according to an embodiment.

FIG. 2 is a side view illustrating the container assembly according to the embodiment.

FIG. 3 is a plan view illustrating the container assembly according to the embodiment.

FIG. 4 is a perspective view illustrating the container assembly according to the embodiment.

FIG. 5 is a sectional view taken along the line A-A in FIG. 3 of the container assembly according to the embodiment.

FIG. 6 is a sectional view taken along the line C-C in FIG. 3 of the container assembly according to the embodiment.

FIGS. 7A and 7B are views schematically illustrating an operation of the container assembly according to the embodiment.

FIGS. 8A and 8B are views schematically illustrating the operation of the container assembly according to the embodiment.

FIG. 9 is a configuration view schematically illustrating a PCR device.

FIG. 10 is a block diagram of the PCR device.

FIG. 11 is a perspective view illustrating a third washing container.

FIG. 12 is a longitudinal sectional view illustrating the third washing container.

FIG. 13 is a longitudinal sectional view illustrating an elution container.

FIG. 14 is a longitudinal sectional view illustrating the third washing container and the elution container.

FIG. 15 is a perspective view illustrating the elution container.

FIG. 16 is a front view illustrating the elution container.

FIGS. 17A to 17F are sectional views of the elution container.

FIG. 18 is a longitudinal sectional view illustrating the third washing container and the elution container.

FIG. 19 is a sectional view taken along the line C-C in FIG. 3 of the container assembly according to the embodiment.

### DESCRIPTION OF EXEMPLARY EMBODIMENTS

Hereinafter, preferred embodiments of the invention will be described in detail with reference to the accompanying drawings. Further, the embodiments described below are not intended to wrongfully limit the contents of the invention described in the aspects of the invention. In addition, all configuration described below are not necessarily indispensable constituent elements of the invention.

A nucleic acid purification device according to the invention is configured such that a washing container in which a washing solution and a fluid which is not mixed with the washing solution are sealed by and stored in and an elution container in which an eluate and a fluid which is not mixed with the eluate are sealed by and stored in are bonded to each other to form a channel for moving a substance (nucleic acid), the washing solution is a liquid which washes a substance-binding solid phase carrier (nucleic acid-binding solid phase carrier) to which the nucleic acid is adsorbed, the eluate is a liquid which separates the nucleic acid from the nucleic acid-binding solid phase carrier, the washing container includes an outer peripheral wall which is arranged by being spaced apart from the channel of the elution container and accommodates a connection portion of the channel of the washing container and the channel of the elution container, the elution container includes a plurality of flanges which is arranged in contact with an inner wall of the outer peripheral wall, and one space which is partitioned by flanges adjacent to each other among the plurality of flanges and the outer peripheral wall communicates with another space adjacent to the one space in a state of being divided by one of the two flanges adjacent to each other.

Examples of living body-related substances include biopolymers as substance related to a living body such as nucleic acids (DNA and RNA), polypeptides, proteins, and polysaccharides; low-molecular organic compounds derived from a living body such as proteins, enzymes, peptides, nucleotides, amino acids, and vitamins; and inorganic compounds. In embodiments described below, the living body-related substances will be described using nucleic acids.

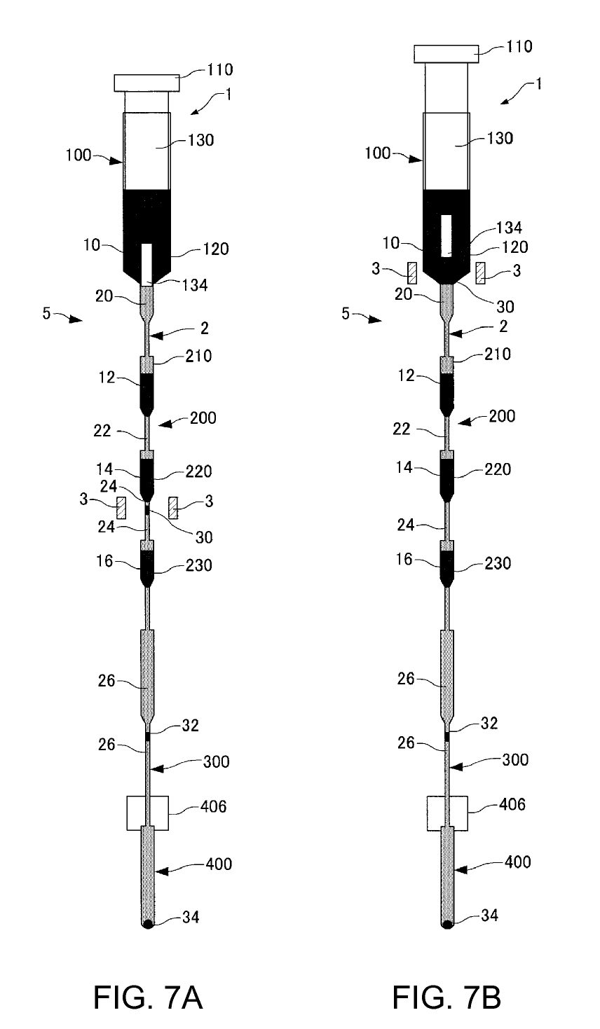

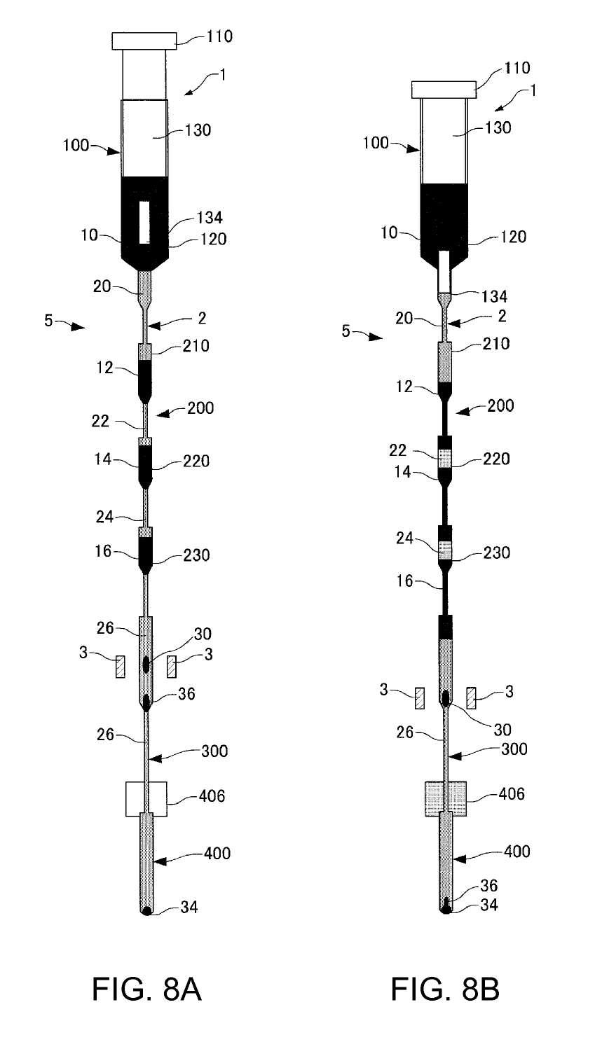

In addition, the substance-binding solid phase carrier is a substance capable of holding a living body-related substance through adsorption, that is, reversible physical bonding. It is preferable that the substance-binding solid phase carrier is in the form of fine particles, but, without being particularly limited thereto, may be in the form of fine fibers or a mesh-like body. It is preferable that the substance-binding solid phase carrier has magnetism because the inside of an assembly is moved to a desired direction in a state in which a living body-related substance is adsorbed. In the embodiments described below, the substance-binding solid phase carrier will be described using magnetic beads 30 (see FIGS. 7A to 8B) that adsorb a nucleic acid.

Washing solutions 12, 14, and 16 (see FIGS. 7A to 8B) are liquids for washing the substance-binding solid phase carrier to which a living body-related substance is adsorbed. Accordingly, by washing the substance-binding solid phase carrier using the washing solutions, other impurities can be removed while allowing the living body-related substance adsorbed to the substance-binding solid phase carrier to be more reliably adsorbed thereto.

The fluid which is not mixed with the washing solution is not mixed with the washing solution in the washing container and is capable of phase separation from the washing solution. The fluid which is not mixed with the washing solution is a substance inert with respect to the washing solution and contains a gas such as the air. In a case where the washing solution is an aqueous liquid, for example, an oil or an oil gel which is not mixed with the aqueous liquid can be used as the fluid which is not mixed with the washing solution. The oil gel is a substance obtained by gelling a liquid-like oil using a gelling agent. Further, in the present embodiment, a gelled substance is excluded at the time of simply referring to as an “oil.” In the embodiments described below, the fluids which are not mixed with the washing solutions will be described using oils 20, 22, 24, and 26 (see FIGS. 7A to 8B described below).

An eluate 32 (see FIGS. 7A to 8B) is obtained by eluting a living body-related substance into an eluate by separating the living body-related substance from the substance-binding solid phase carrier. As the eluate, for example, water or a buffer solution can be used.

The fluid which is not mixed with the eluate is not mixed with the eluate in the elution container and is capable of phase separation from the washing solution. The fluid which is not mixed with the eluate is a substance inert with respect to the eluate. In the embodiments described below, the fluid which is not mixed with the washing solution will be described using the oil 26 (see FIGS. 7A to 8B described below).

### 1. Outline of Container Assembly

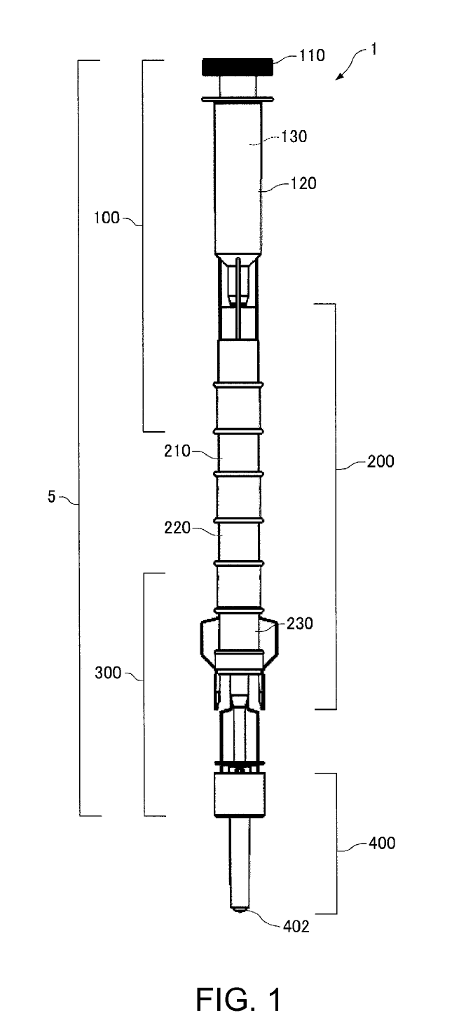

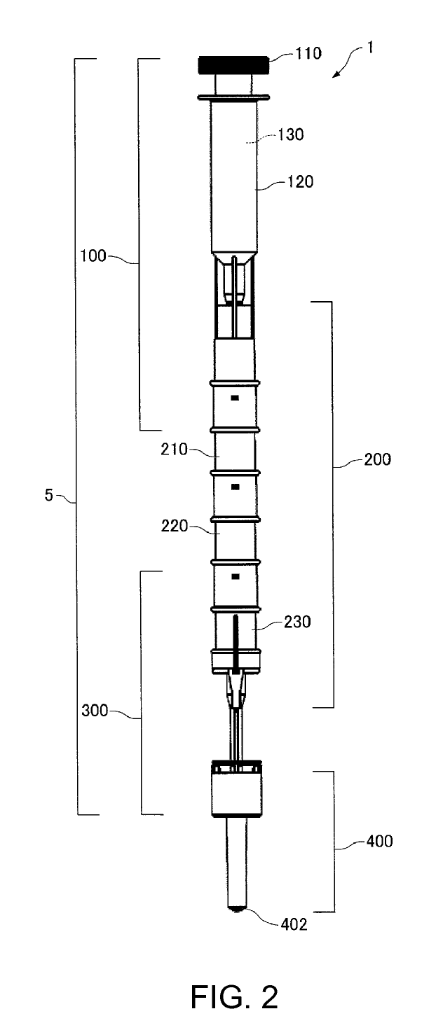

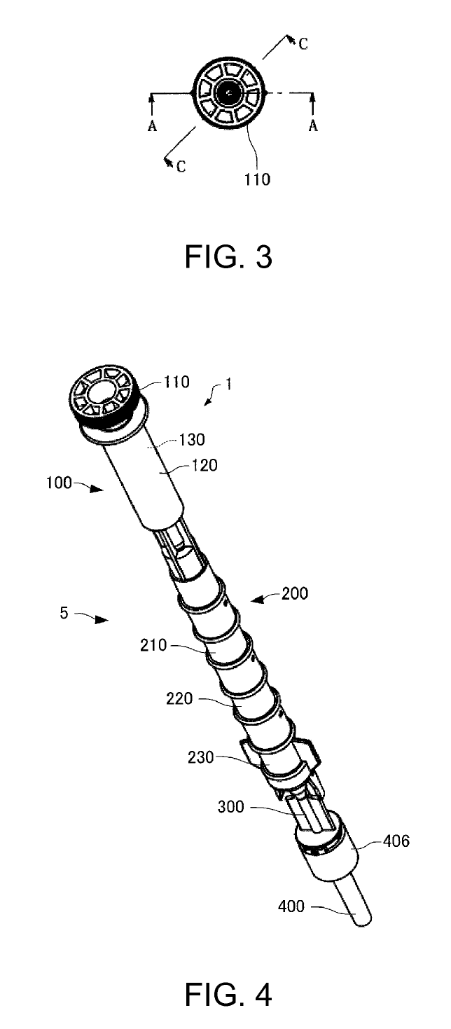

First, the outline of a container assembly 1 according to the present embodiment will be described with reference to FIGS. 1 to 4. FIG. 1 is a front view illustrating the container assembly 1 (hereinafter, also referred to as a cartridge) according to the embodiment. FIG. 2 is a side view illustrating the container assembly 1 according to the embodiment. FIG. 3 is a plan view illustrating the container assembly 1 according to the embodiment. FIG. 4 is a perspective view illustrating the container assembly 1 according to the embodiment. In FIGS. 1 to 3, the container assembly 1 is described to be in an erected state.

The container assembly 1 includes an adsorption container 100, a washing container 200, an elution container 300, and a reaction container 400. The container assembly 1 is a container that forms a channel (not illustrated) communicating from the adsorption container 100 to the reaction container 400. In the channel of the container assembly 1, one end portion is closed by a cap 110 and another end portion is closed by a bottom portion 402.

The container assembly 1 is a container in which a pre-treatment of bonding a nucleic acid to a magnetic bead (not illustrated) in the adsorption container 100, purifying the nucleic acid while the magnetic bead is moved in the washing container 200, and eluting the nucleic acid into an eluate droplet (not illustrated) in the elution container 300 and a thermal cycle treatment of causing a polymerase reaction with respect to the droplet of the eluate containing the nucleic acid in the reaction container 400 are performed.

As the material of the container assembly 1, which is not particularly limited, glass, a polymer, or a metal can be used. It is more preferable that a material having transparency with respect to visible light, for example, glass or a polymer is selected as the material of the container assembly 1 because the inside (in a cavity) can be observed from the outside of the container assembly 1. It is preferable that a substance transmitting magnetic force or a non-magnetic material is selected as the material of the container assembly 1 because magnetic beads (not illustrated) can easily pass through the container assembly 1 by applying magnetic force from the outside of the container assembly 1. For example, a polypropylene resin can be used as the material of the container assembly 1.

The adsorption container 100 includes a cylindrical syringe portion 120 that accommodates an adsorption solution (not illustrated) in the inside thereof, a plunger portion 130 which is a movable plunger inserted into the inside of the syringe portion 120, and the cap 110 which is to be fixed to one end portion of the plunger portion 130. The adsorption container 100 can allow the plunger portion 130 to slide on the inner surface of the syringe portion 120 by moving the cap 110 with respect to the syringe portion 120 such that an adsorption solution (not illustrated) accommodated in the syringe portion 120 pushes the washing container 200. In addition, the adsorption solution will be described below.

The washing container 200 can be obtained by bonding and assembling first to third washing containers 210, 220, and 230. The first to third washing containers 210, 220, and 230 respectively include one or more washing solution layers which are partitioned by an oil layer (not illustrated) in the inside thereof. Further, the washing container 200 includes a plurality of washing solution layers partitioned by a plurality of oil layers (not illustrated) in the inside thereof by bonding the first to third washing containers 210, 220, and 230. In the washing container 200 of the present embodiment, the example using three washing containers formed of the first to third washing containers 210, 220, 230 has been described, but, without being limited thereto, the number of washing containers can be appropriately increased or decreased according to the number of washing solution layers. Further, the washing solution will be described below.

The elution container 300 is bonded to the third washing container 230 of the washing container 200 and accommodates an eluate in the inside thereof in a state in which the form of a plug can be maintained. Here, the term “plug” indicates a liquid in a case where a particular liquid occupies one section in a channel. More specifically, the plug of the particular liquid indicates a columnar liquid in which only the particular liquid substantially occupies the inside in the longitudinal direction of the channel in a state in which a certain space in the inside of the channel is partitioned by the plug of the liquid. Here, the expression “substantially” above indicates that a small amount (for example, thin film-like) of another substance (liquid or the like) may be present in the periphery of the plug, that is, the inner wall of the channel. Further, the eluate will be described below.

A nucleic acid purification device 5 includes the adsorption container 100, the washing container 200, and the elution container 300.

The reaction container 400 is a container which is bonded to the elution container 300, receives a liquid pushed from the elution container 300, and accommodates droplets of an eluate containing a specimen at the time of the thermal cycle treatment. In addition, the reaction container 400 accommodates a reagent (not illustrated). Further, the reagent will be described below.

### 2. Detailed Structure of Container Assembly

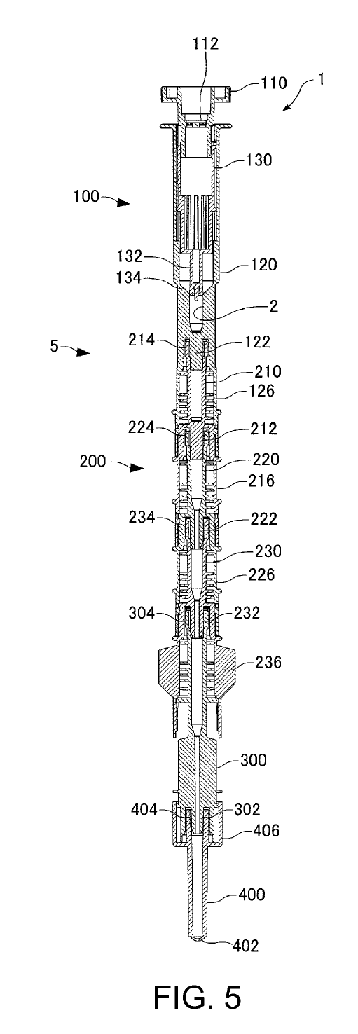

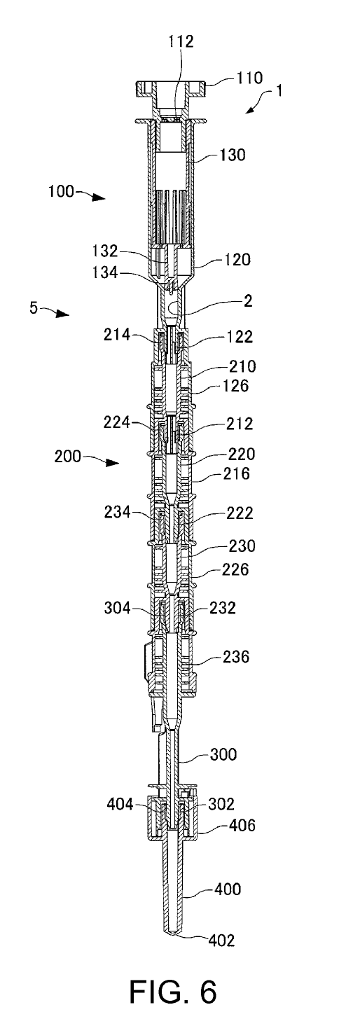

Next, detailed structure of the container assembly 1 will be described with reference to FIGS. 5 and 6. FIG. 5 is a sectional view taken along the line A-A in FIG. 3 of the container assembly 1 according to the embodiment. FIG. 6 is a sectional view taken along the line C-C in FIG. 3 of the container assembly 1 according to the embodiment. Further, practically, the container assembly 1 is assembled in a state in which the contents such as a washing solution are filled, but the description of the contents is not made in FIGS. 5 and 6 for description of the structure of the container assembly 1.

### 2-1. Adsorption Container

The plunger portion 130 is inserted to the adsorption container 100 from one opening end portion of the syringe portion 120 and the cap 110 is inserted to an opening end portion of the plunger portion 130. The cap 110 includes a ventilation portion 112 in the center thereof and a change in the internal pressure of the plunger portion 130 can be suppressed by the ventilation portion 112 at the time of operating the plunger portion 130.

The plunger portion 130 is an approximately cylindrical plunger that slides on the inner peripheral surface of the syringe portion 120 and includes an opening end portion to which the cap 110 is inserted, a rod-like portion 132 that extends in the longitudinal direction of the syringe portion 120 from the bottom portion facing the opening end portion, and a tip portion 134 positioned on the tip of the rod-like portion 132. The rod-like portion 132 is projected from the center of the bottom portion of the plunger portion 130 and through holes are formed in the periphery of the rod-like portion 132 such that the inside of the plunger portion 130 communicates with the inside of the syringe portion 120.

The syringe portion 120 constitutes a part of a channel 2 of the container assembly 1 and includes a large-diameter portion which accommodates the plunger portion 130, a small-diameter portion whose inner diameter is smaller than that of the large-diameter portion, a reduced-diameter portion which reduces the inner diameter from the large-diameter portion to the small-diameter portion, an adsorption insertion portion 122 to which the tip of the small-diameter portion is adsorbed and inserted, and a cylindrical adsorption cover portion 126 which covers the periphery of the adsorption insertion portion 122. The large-diameter portion and the small-diameter portion which become a part of the channel 2 of the container assembly 1 and the adsorption insertion portion 122 are approximately cylindrical.

When the container assembly 1 is provided to an operator, the tip portion 134 of the plunger portion 130 seals the small-diameter portion of the syringe portion 120 to partition the large-diameter portion, the reduced-diameter portion, and the small-diameter portion and forms two sections.

The adsorption insertion portion 122 of the syringe portion 120 bonds the syringe portion 120 to the first washing container 210 by being inserted and fitted to a first reception portion 214 which is one opening end portion of the first washing container 210 in the washing container 200. The leakage of a liquid, which is the content, to the outside is prevented by bringing the outer peripheral surface of the adsorption insertion portion 122 into close contact with the inner peripheral surface of the first reception portion 214.

### 2-2. Washing Container

The washing container 200 is an assembly that constitutes a part of the channel 2 of the container assembly 1 and is formed of the first to third washing containers 210, 220, and 230. Since the basic structures of the first to third washing containers 210, 220, and 230 are the same as each other, the structure of the first washing container 210 is described and the description of the second and third washing containers 220 and 230 is not repeated.

The first washing container 210 is an approximately cylindrical container extending in the longitudinal direction of the container assembly 1 and includes a first insertion portion 212 formed on one opening end portion, a first reception portion 214 formed on another opening end portion, and a cylindrical first cover portion 216 covering the periphery of the first insertion portion 212.

The outer diameter of the first insertion portion 212 is approximately the same as the inner diameter of a second reception portion 224. Further, the inner diameter of the first reception portion 214 is approximately the same as the outer diameter of the adsorption insertion portion 122.

When the first insertion portion 212 of the first washing container 210 is inserted and fitted to the second reception portion 224 of the second washing container 220, the outer periphery of the first insertion portion 212 is brought into close contact with the inner periphery of the second reception portion 224 to be sealed and the first washing container 210 and the second washing container 220 are bonded to each other. In the same manner, the first to third washing containers 210, 220, and 230 are connected to one another to form the washing container 200. Here, the expression “to be sealed” indicates sealing such that a liquid or a gas accommodated at least in a container or the like is not leaked to the outside and may include sealing such that a liquid or a gas is not intruded to the inside from the outside.

### 2-3. Elution Container

The elution container 300 is an approximately cylindrical container extending in the longitudinal direction of the container assembly 1 and configures apart of the channel 2 of the container assembly 1. The elution container 300 includes an elution insertion portion 302 formed on one opening end portion and an elution reception portion 304 formed on another opening end portion.

The inner diameter of the elution reception portion 304 is approximately the same as the outer diameter of a third insertion portion 232 of the third washing container 230. When the third insertion portion 232 is inserted and fitted to the elution reception portion 304, the outer periphery of the third insertion portion 232 is brought into close contact with the inner periphery of the elution reception portion 304 to be sealed and the third washing container 230 and the elution container 300 are bonded to each other.

### 2-4. Reaction Container

The reaction container 400 is an approximately cylindrical container extending in the longitudinal direction of the container assembly 1 and constitutes a part of the channel 2 of the container assembly 1. The reaction container 400 includes a reaction reception portion 404 formed on an opening end portion, a bottom portion 402 formed on another closed end portion, and a reservoir portion 406 covering the reaction reception portion 404.

The inner diameter of the reaction reception portion 404 is approximately the same as the outer diameter of the elution insertion portion 302 of the elution container 300. When the elution insertion portion 302 is inserted and fitted to the reaction reception portion 404, the elution container 300 and the reaction container 400 are bonded to each other.

The reservoir portion 406 having a predetermined space is provided in the periphery of the reaction reception portion 404. The reservoir portion 406 has a volume that can receive a liquid overflowing from the reaction container 400 because of the movement of the plunger portion 130.

### 3. Contents of Container Assembly and Operation of Container Assembly

Next, the contents of the container assembly 1 will be described with reference to FIG. 7A and the operation of the container assembly 1 will be described with reference to FIGS. 7A to 8B. FIGS. 7A and 7B are views schematically illustrating the operation of the container assembly 1 according to the embodiment. FIGS. 8A and 8B are views schematically illustrating the operation of the container assembly 1 according to the embodiment. In addition, since FIGS. 7A to 8B describe the state of the contents, respective containers are expressed using the channel 2 and the external shape or the bonding structure thereof will not be described.

### 3-1. Contents

FIG. 7A illustrates the state of the contents in the channel 2 in the state of FIG. 1. The contents in the channel 2 are an adsorption solution 10, a first oil 20, a first washing solution 12, a second oil 22, a second washing solution 14, a third oil 24, a magnetic bead 30, a third oil 24, a third washing solution 16, a fourth oil 26, an eluate 32, a fourth oil 26, and a reagent 34 in order toward the reaction container 400 from the cap 110 side.

In the channel 2, a portion (thick portion of the channel 2) whose sectional area of a surface perpendicular to the longitudinal direction of the container assembly 1 is large and a portion (thin portion of the channel 2) whose sectional area thereof is small are alternately arranged. Some or all of the first to fourth oils 20, 22, 24, and 26 and the eluate 32 are accommodated in the thin portion of the channel 2. In a case where the interface between liquids (or fluids, the same applies to hereinafter) which are adjacent to each other and not mixed with each other is arranged in the thin portion of the channel 2, the sectional area of the thin portion of the channel 2 has an area in which the interface can be stably maintained. Therefore, an arrangement relationship between the liquids and other liquids arranged on and below the liquids can be stably maintained by the liquids arranged in the thin portion of the channel 2. In addition, even in a case where the interface between a liquid arranged in the thin portion of the channel 2 and another liquid arranged in the thick portion of the channel 2 is formed in the thick portion of the channel 2, the interface is stably formed in a predetermined position by being placed in a stationary state even when the interface is disturbed due to a strong impact.

The thin portion of the channel 2 is formed in the inside of the adsorption insertion portion 122, the first insertion portion 212, the second insertion portion 222, the third insertion portion 232, and the elution insertion portion 302 and extends to the upper portion beyond the elution insertion portion 302 in the elution container 300. In addition, the liquid accommodated in the thin portion of the channel 2 is stably maintained even before the container is assembled.

### 3-1-1. Oils

All of the first to fourth oils 20, 22, 24, and 26 are formed of oils and exist as plugs between liquids in front and behind of respective oils in the state of FIGS. 7A and 7B. since the first to fourth oils 20, 22, 24, and 26 exist as plugs, liquids which are phase-separated from each other, that is, liquids which are not mixed with each other are selected as the liquids adjacent to each other in front and behind of respective oils. The oils constituting the first to fourth oils 20, 22, 24, and 26 may be oils different from each other. As oils which can be used as the oils, a silicone-based oil such as a dimethyl silicone oil, a paraffin-based oil, a mineral oil, and an oil selected from mixtures of those can be exemplified.

### 3-1-2. Adsorption Solution

The adsorption solution 10 indicates a liquid which becomes a place that allows the magnetic bead 30 to adsorb a nucleic acid and is, for example, an aqueous solution containing a chaotropic substance. As the adsorption solution 10, for example, 5M guanidine thiocyanate, 2% Triton X-100, or 50 mM Tris-HCl (pH 7.2) can be used. The adsorption solution 10 is not particularly limited as long as the adsorption solution contains a chaotropic substance, but the adsorption solution 10 may contain a surfactant for the purpose of destroying a cell membrane or modifying proteins contained in a cell. The surfactant is not particularly limited as long as the surfactant is used for extracting a nucleic acid from a cell or the like and examples thereof include a triton-based surfactant such as Triton-X, a non-ionic surfactant, for example, a tween-based surfactant such as Tween 20, and an anionic surfactant such as sodium N-lauroylsarcosine (SDS). Particularly, it is preferable that a non-ionic surfactant is contained in the range of 0.1% to 2%. Further, it is preferable that a reducing agent such as 2-mercapto ethanol or dithiothreitol is contained. A solution may be a buffer solution and is preferably neutral with a pH of 6 to 8. In consideration of these, specifically, 3M to 7M guanidine salts, 0% to 5% of a non-ionic surfactant, 0 mM to 0.2 mM of EDTA, and 0 M to 0.2 M of a reducing agent are preferably contained.

Here, the chaotropic substance is not particularly limited as long as chaotropic ions (monovalent anions which are large in ionic radius) are generated in an aqueous solution and the chaotropic substance has an action of increasing water solubility of a hydrophobic molecule and contributes to adsorption of a nucleic acid to a solid phase carrier. Specific examples thereof include guanidine hydrochloride, sodium iodide, and sodium perchlorate. Among these, guanidine thiocyanate or guanidine hydrochloride having a strong protein metamorphism is preferable. The specification concentrations of these chaotropic substances are different from each other according to respective substance and it is preferable that 3 M to 5.5 M of guanidine thiocyanate is used or 5 M or more of guanidine hydrochloride is used.

When the chaotropic substance exists in an aqueous solution, since it is thermodynamically favorable for a nucleic acid in the aqueous solution to exist by being adsorbed to a solid rather than a case where the nucleic acid exists by being surrounded by water molecules, the nucleic acid is to be adsorbed to the surface of the magnetic bead 30.

### 3-1-3. Washing Solution

The first to third washing solutions 12, 14, and 16 are solutions that wash the magnetic bead 30 bonded to a nucleic acid.

The first washing solution 12 is a liquid that is phase-separated from both of the first oil 20 and the second oil 22. It is preferable that the first washing solution 12 is water or a low salt concentration aqueous solution and the low salt concentration aqueous solution is a buffer solution. The salt concentration of the low salt concentration aqueous solution is preferably 100 mM or less, more preferably 50 mM or less, and most preferably 10 mM or less. In addition, the first washing solution 12 may contain a surfactant as described above and the pH thereof is not particularly limited. Salts for using the first washing solution 12 as a buffer solution are not particularly limited, and preferable examples thereof include tris, hepes, pipes, and phosphoric acid. Further, it is preferable that the first washing solution 12 contains alcohol in an amount in which adsorption of a nucleic acid to a carrier, a reverse transcription reaction or a PCR reaction is not inhibited. In this case, the alcohol concentration is not particularly limited.

In addition, the first washing solution 12 may contain a chaotropic substance. For example, when the first washing solution 12 contains guanidine hydrochloride, the magnetic bead 30 or the like can be washed while adsorption of a nucleic acid which is adsorbed to the magnetic bead 30 or the like is maintained or strengthened.

The second washing solution 14 is a liquid that is phase-separated from both of the second oil 22 and the third oil 24. The second washing solution 14 may have a composition which is the same as or different from that of the first washing solution 12, but is preferably a solution that does not substantially contain a chaotropic substance so that the chaotropic substance is not taken by the subsequent solution. The second washing solution 14 may be formed of, for example, a 5 mM tris hydrochloric acid buffer solution. As described above, it is preferable that the second washing solution 14 contains alcohol.

The third washing solution 16 is a liquid that is phase-separated from both of the third oil 24 and the fourth oil 26. The third washing solution 16 may have a composition which is the same as or different from that of the second washing solution 14, but does not contain alcohol. In addition, the third washing solution 16 can contain citric acid to prevent alcohol from being taken by the reaction container 400.

### 3-1-4. Magnetic Bead

The magnetic bead 30 is a bead that adsorbs a nucleic acid and preferably has relatively strong magnetism such that the bead is moved by a magnet 3 positioned out of the container assembly 1. For example, the magnetic bead 30 may be a silica bead or a bead coated with silica. The magnetic bead 30 may be preferably a bead coated with silica.

### 3-1-5. Eluate

The eluate 32 is a liquid which is phase-separated from the fourth oil 26 and exists as a plug interposed by the fourth oils 26 and 26 in the channel 2 of the elution container 300. The eluate 32 is a liquid that elutes a nucleic acid adsorbed to the magnetic bead 30 into the eluate 32 from the magnetic bead 30. Further, the eluate 32 becomes droplets in the fourth oil 26 due to heating. For example, pure water can be used as the eluate 32. Here, the “droplet” is a liquid surrounded by a free surface.

### 3-1-6. Reagent

The reagent 34 contains components necessary for a reaction. In a case where the reaction in the reaction container 400 is the PCR, the reagent 34 can contain at least one from among enzymes and primers (nucleic acid) such as DNA polymerase for amplifying a target nucleic acid (DNA) eluted into a droplet 36 (see FIGS. 8A and 8B) of the eluate and a fluorescent probe for detecting an amplified product. Here, the reagent 34 contains all of primers, enzymes, and a fluorescent probe. The reagent 34 is not compatible with the fourth oil 26, reacted by being melted when a nucleic acid is brought into contact with the droplet 36 of the eluate 32, and exists in a region of the lowermost portion in the gravity direction of the channel 2 in the reaction container 400 in a solid state. For example, a reagent which is freeze-dried can be used as the reagent 34.

### 3-2. Operation of Container Assembly

An example of the operation of the container assembly 1 will be described with reference to FIGS. 7A to 8B.

The operation of the container assembly 1 includes (A) a process of assembling the container assembly 1 by bonding the adsorption container 100, the washing container 200, the elution container 300, and the reaction container 400; (B) a process of introducing a specimen containing a nucleic acid to the adsorption container 100 accommodating the adsorption solution 10; (C) a process of moving the magnetic bead 30 to the adsorption container 100 from the second washing container 220; (D) a process of allowing the nucleic acid to be adsorbed to the magnetic bead 30 by swinging the adsorption container 100; (E) a process of moving the magnetic bead 30 to which the nucleic acid is adsorbed to the elution container 300 from the adsorption container 100 by allowing the magnetic bead 30 to pass through the first oil 20, the first washing solution 12, the second oil 22, the second washing solution 14, the third oil 24, the third washing solution 16, and the fourth oil 26 in this order; (F) a process of eluting the nucleic acid from the magnetic bead 30 with respect to the eluate 32 in the elution container 300; and (G) a process of bringing a droplet containing the nucleic acid into contact with the reagent 34 in the reaction container 400.

Hereinafter, respective processes will be sequentially described.

### A. Process of Assembling Container Assembly 1

As illustrated in FIG. 7A, the process of assembling the container assembly 1 is carried out by assembling the container assembly 1 such that the channel 2 in which the adsorption container 100 to the reaction container 400 are continued is formed by bonding the adsorption container 100 to the reaction container 400 to one another. In addition, in FIG. 7A, the cap 110 is mounted on the adsorption container 100, and the cap 110 is mounted on the plunger portion 130 after the process (B).

More specifically, the elution insertion portion 302 of the elution container 300 is inserted into the reaction reception portion 404 of the reaction container 400, the third insertion portion 232 of the third washing container 230 is inserted into the elution reception portion 304 of the elution container 300, the second insertion portion 222 of the second washing container 220 is inserted into a third reception portion 234 of the third washing container 230, the first insertion portion 212 of the first washing container 210 is inserted into the second reception portion 224 of the second washing container 220, and the adsorption insertion portion 122 of the adsorption container 100 is inserted into the first reception portion 214 of the first washing container 210.

### B. Process of Introducing Specimen

The process of introducing a specimen is carried out by inserting a cotton swab to which the specimen is attached into the adsorption solution 10 from an opening on which the cap 110 of the adsorption container 100 is mounted and immersing the cotton swab in the adsorption solution 10. More specifically, the cotton swab is inserted from the opening positioned in one end portion of the plunger portion 130 in a state of being inserted into the syringe portion 120 of the adsorption container 100. Next, the cotton swab is taken out of the adsorption container 100 and the cap 110 is mounted on the opening. This state is illustrated in FIG. 7A. In addition, the specimen may be introduced to the adsorption container 100 by a pipette or the like. Further, when the specimen is in the form of paste or a solid, the specimen may be attached to the inner wall of the plunger portion 130 or input to the adsorption container 100 using a spoon or tweezers. As illustrated in FIG. 7A, the syringe portion 120 and the plunger portion 130 are filled with the adsorption solution 10 up to the halfway thereof, but a space remains on the opening side on which the cap 110 is mounted.

The specimen contains a nucleic acid serving as a target. Hereinafter, the nucleic acid is also simply referred to as a target nucleic acid. The target nucleic acid is deoxyribonucleic acid (DNA) or ribonucleic acid (RNA). After the target nucleic acid is extracted from the specimen and eluted into the eluate 32 described below, the target nucleic acid is used as, for example, a mold of the PCR. Examples of the specimen include blood, nasal mucus, oral mucosa, and other kinds of biological samples.

### C. Process of Moving Magnetic Bead

The process of moving the magnetic bead 30 is carried out by moving the magnet 3 toward the adsorption container 100 in a state in which the magnetic force of the magnet 3 arranged outside of the container is applied to the magnetic bead 30 present in the form of a plug which is interposed by the third oils 24 and 24 of the second washing container 220 as illustrated in FIG. 7A.

The cap 110 and the plunger portion 130 are moved to the direction of extraction from the syringe portion 120 simultaneously with or before the movement of the magnetic bead 30 and then the specimen in the adsorption solution 10 is moved to the inside of the syringe portion 120 from the inside of the plunger portion 130. The channel 2 blocked by the tip portion 134 communicates with the adsorption solution 10 by the movement of the plunger portion 130.

The magnetic bead 30 is lifted in the channel 2 along the movement of the magnet 3 and reaches inside of the adsorption solution 10 including the specimen as illustrated in FIG. 7B.

### D. Process of Adsorbing Nucleic Acid to Magnetic Bead

The process of adsorbing the nucleic acid is carried out by swinging the adsorption container 100. This process can be efficiently performed since the opening of the adsorption container 100 is sealed by the cap 110 such that the adsorption solution 10 is not leaked. By performing the process, the target nucleic acid is adsorbed to the surface of the magnetic bead 30 due to the action of a chaotropic agent. In this process, a nucleic acid or proteins instead of the target nucleic acid may be adsorbed to the surface of the magnetic bead 30.

As a method of swinging the adsorption container 100, a device such as a known vortex shaker or the like may be used or the adsorption container 100 may be manually swung by an operator. In addition, the adsorption container 100 may be swung while a magnetic field is provided from the outside using the magnetism of the magnetic bead 30.

E. Process of Moving Magnetic Bead to which Nucleic Acid is Adsorbed

The process of moving the magnetic bead 30 to which the nucleic acid is adsorbed is carried out by moving the magnetic bead 30 while the magnetic force of the magnet 3 is applied from the outside of the adsorption container 100, the washing container 200, and the elution container 300 such that the magnetic bead 30 is allowed to pass through the adsorption solution 10, the first to fourth oils 20, 22, 24, and 26 and the first to third washing solutions 12, 14, and 16.

For example, a permanent magnet or an electromagnet can be used as the magnet 3. Further, the magnet 3 may be used by being manually moved by the operator or using a mechanical device or the like. Since the magnetic bead 30 has a property of being drawn by the magnetic force, the magnetic bead 30 is moved to the adsorption container 100, the washing container 200, and the elution container 300 in the channel 2 by changing the relative arrangement of the magnet 3 using the property. The speed at the time when the magnetic bead 30 passes through the respective washing solutions is not particularly limited and the magnetic bead 30 may be moved so as to reciprocate along the longitudinal direction of the channel 2 in the same washing solution. Further, in a case where particles or the like other than the magnetic bead 30 are moved in a tube, the particles or the like can be moved using the gravity or a potential difference.

### F. Process of Eluting Nucleic Acid

The process of eluting the nucleic acid is carried out by eluting the nucleic acid from the magnetic bead 30 with respect to the droplet 36 of the eluate in the elution container 300. The eluate 32 in FIGS. 7A and 7B exists as a plug in the thin portion of the channel of the elution container 300, but the eluate 32 vertically moves in the elution container 300 as the droplet 36 as illustrated in FIGS. 8A and 8B after the content liquid is expanded by heating the reaction container 400 while the magnetic bead 30 is moved as described above. Moreover, as illustrated in FIG. 8A, when the magnetic bead 30 reaches the droplet 36 of the eluate of the elution container 300, the target nucleic acid adsorbed to the magnetic bead 30 is eluted into the droplet 36 of the eluate due to the action of the eluate.

G. Process of Bringing Droplet 36 into Contact with Reagent 34

The process of bringing the droplet 36 into contact with the reagent 34 is carried out by bringing the droplet 36 containing the nucleic acid into contact with the reagent 34 positioned in the lowermost portion of the reaction container 400. Specifically, as illustrated in FIG. 8B, the droplet 36 of the eluate in which the target nucleic acid is eluted is moved to the reaction container 400 and is brought into contact with the reagent 34 positioned in the lowermost portion of the reaction container 400 while the magnetic bead 30 to which the magnetic force of the magnet 3 is applied is maintained in a predetermined position by pushing the cap 110 and pushing the first oil 20 down using the tip portion 134 of the plunger portion 130. The reagent 34 in contact with the droplet 36 is melted and mixed with the target nucleic acid in the eluate and thus, for example, the PCR using the thermal cycle can be performed.

### 4. PCR Device

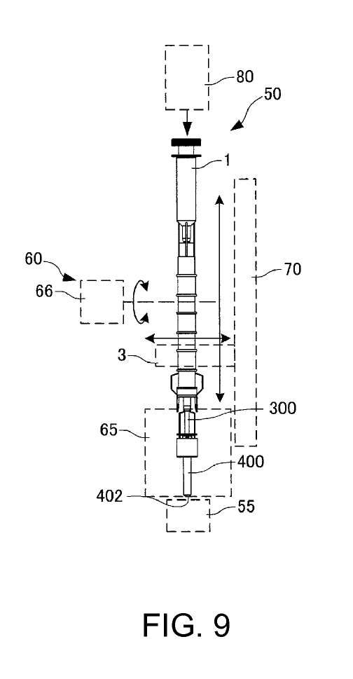

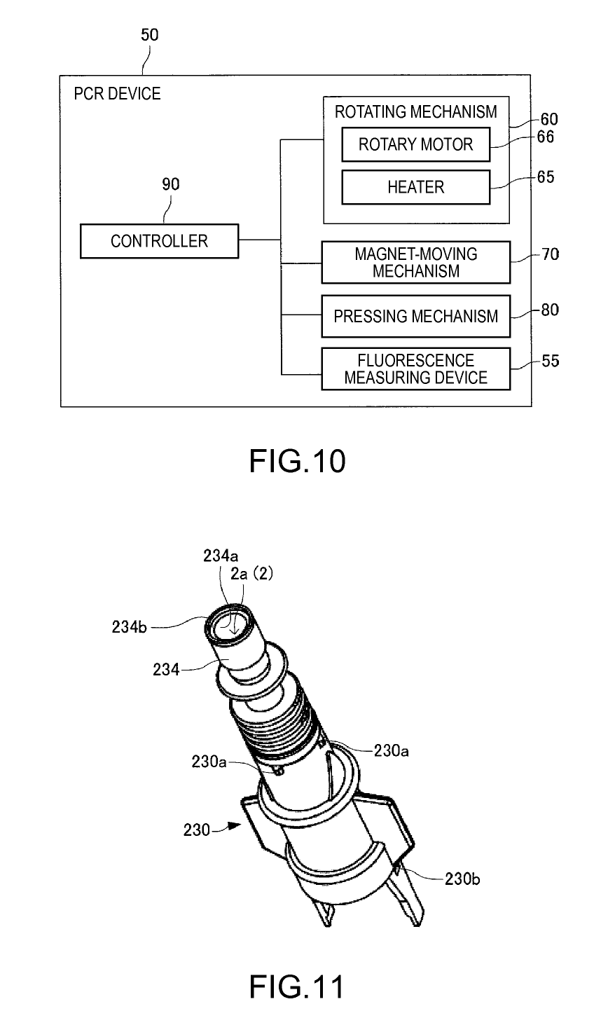

A PCR device 50 that performs a nucleic acid elution treatment and the PCR using the container assembly 1 will be described with reference to FIGS. 9 and 10. FIG. 9 is a configuration view schematically illustrating the PCR device 50. FIG. 10 is a block diagram of the PCR device 50.

The PCR device 50 includes a rotating mechanism 60, a magnet-moving mechanism 70, a pressing mechanism 80, a fluorescence measuring device 55, and a controller 90.

### 4-1. Rotating Mechanism

The rotating mechanism 60 includes a rotary motor 66 and a heater 65 and rotates the container assembly 1 and the heater 65 by driving the rotary motor 66. The droplet containing the target nucleic acid is moved in the channel of the reaction container 400 when the rotating mechanism 60 rotates the container assembly 1 and the heater 65 to be turned upside down, and then the thermal cycle treatment is performed.

The heater 65 can contain a plurality of heaters (not illustrated), for example, heaters for elution, a high temperature, and a low temperature. The heater for elution heats the plug-like eluate of the container assembly 1 and promotes elution of the target nucleic acid into the eluate from the magnetic bead. The heater for a high temperature heats the liquid on the upstream side of the channel in the reaction container 400 at a temperature higher than that of the heater for a low temperature. The heater for a low temperature heats the bottom portion 402 of the channel in the reaction container. A temperature gradient can be formed in the liquid of the channel in the reaction container 400 by the heater for a high temperature and the heater for a low temperature. The heater 65 is provided with a temperature regulator and the liquid in the container assembly 1 can be set to a temperature suitable for the treatment according to an instruction of the controller 90.

The heater 65 includes an opening to which an outer wall of the bottom portion 402 of the reaction container 400 is exposed. The fluorescence measuring device 55 measures the brightness of the droplet of the eluate from the opening.

### 4-2. Magnet-Moving Mechanism

The magnet-moving mechanism 70 is a mechanism for moving the magnet 3. The magnet-moving mechanism 70 moves the magnetic bead in the container assembly 1 by drawing the magnetic bead in the container assembly 1 to the magnet 3 and moving the magnet 3. The magnet-moving mechanism 70 includes a pair of magnets 3, a lifting mechanism, and a swinging mechanism.

The swinging mechanism is a mechanism for swinging the pair of magnets 3 in the lateral direction of FIG. 9 (may be in the longitudinal direction of FIG. 9). The pair of magnets 3 are arranged so as to interpose the container assembly 1 mounted on the PCR device 50 therebetween in the lateral direction (see FIGS. 7 and 8) and can allow the magnetic bead and the magnet 3 to approach each other in a direction (here, in the lateral direction of FIG. 9) perpendicular to the channel of the container assembly 1. Therefore, when the pair of magnets 3 are swung in the lateral direction as indicated by an arrow, the magnetic bead in the container assembly 1 is moved in the lateral direction along the movement. The lifting mechanism can move the magnet 3 in the vertical direction and move the magnetic bead in the vertical direction of FIG. 9 along the movement of the magnet 3.

### 4-3. Pressing Mechanism

The pressing mechanism 80 is a mechanism for pushing the plunger portion of the container assembly 1 and configured such that the PCR is performed in the reaction container 400 when the droplet in the elution container 300 is pushed out into the reaction container 400 by the plunger portion being pushed by the pressing mechanism 80.

FIG. 9 illustrates the pressing mechanism 80 being arranged in the upper portion of the erected container assembly 1, but the direction in which the pressing mechanism 80 pushes the plunger portion may be the vertical direction in FIG. 9 or may be a direction inclined by 45° with respect to the vertical direction. In this manner, the pressing mechanism 80 is easily arranged in a position that does not interfere with the magnet-moving mechanism 70.

### 4-4. Fluorescence Measuring Device

The fluorescence measuring device 55 is a measuring device that measures the brightness of the droplet in the reaction container 400. The fluorescence measuring device 55 is arranged in a position facing the bottom portion 402 of the reaction container 400. Further, it is desired that the fluorescence measuring device 55 can detect the brightness of a plurality of wavelength regions so as to correspond to the multiplex PCR.

### 4-5. Controller

The controller 90 is a control unit that performs control of the PCR device 50. The controller 90 includes a processor such as a CPU and a storage device such as a ROM or a RAM. The storage device stores various programs and data. Further, the storage device provides a region that develops programs. Various processes are realized by the processor executing the programs stored in the storage device.

For example, the controller 90 controls the rotary motor 66 and rotates the container assembly 1 to a predetermined rotation position. The rotation mechanism 60 is provided with a rotation position sensor (not illustrated) and the controller 90 drives or stops the rotary motor 66 according to the detection results of the rotation position sensor.

Further, the controller 90 controls the heater 65, performs on/off control on the heater 65 to generate heat, and allows the heater 65 to heat the liquid in the container assembly 1 to a predetermined temperature.

In addition, the controller 90 controls the magnet-moving mechanism 70, moves the magnet 3 in the vertical direction, and swings the magnet 3 in the lateral direction of FIG. 9 according to the detection results of the position sensor (not illustrated).

Further, the controller 90 controls the fluorescence measuring device 55 and measures the brightness of the droplet in the reaction container 400. The measurement results are stored in the storage device (not illustrated) of the controller 90.

The processes (C) to (G) of the section 3-2 described above can be performed by mounting the container assembly 1 on the PCR device 50 and the PCR can be further performed.

### 5. Detailed Structure of Nucleic Acid Purification Device

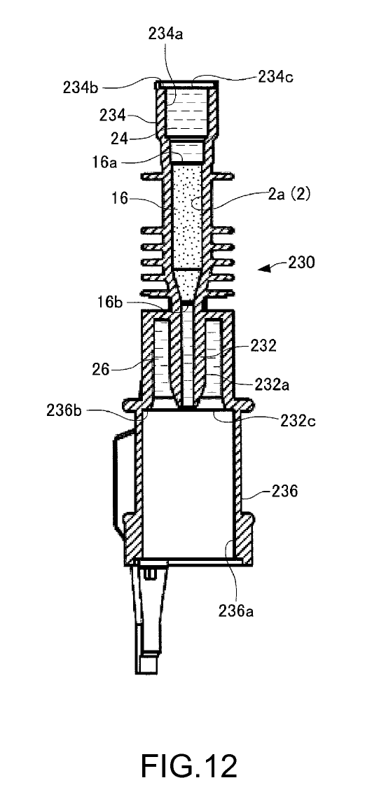

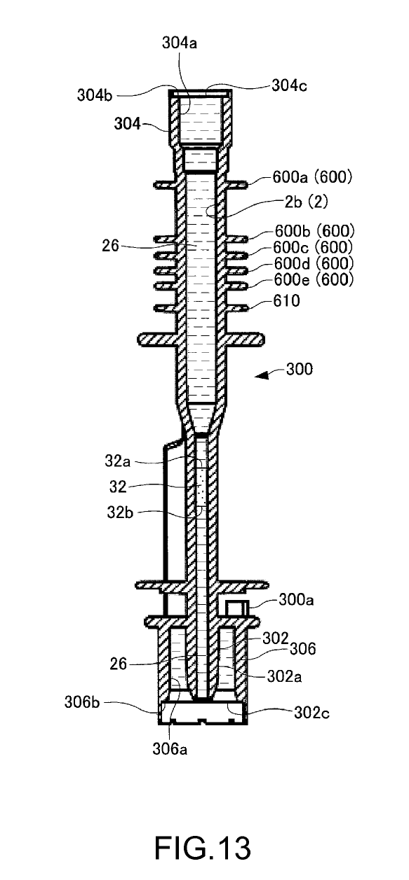

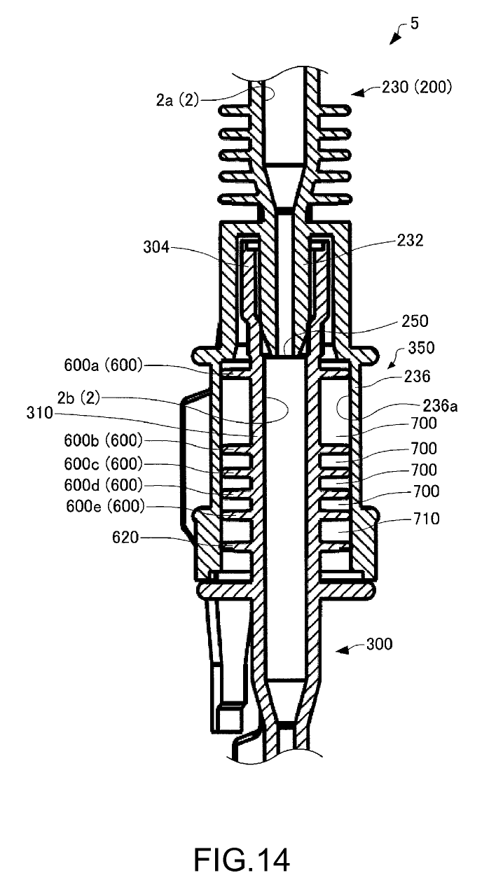

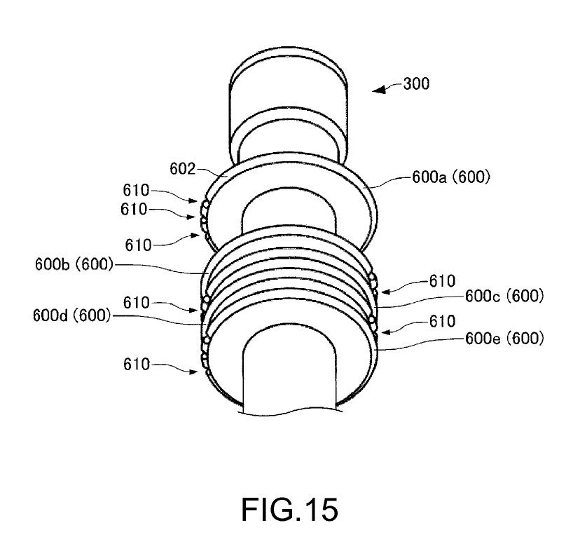

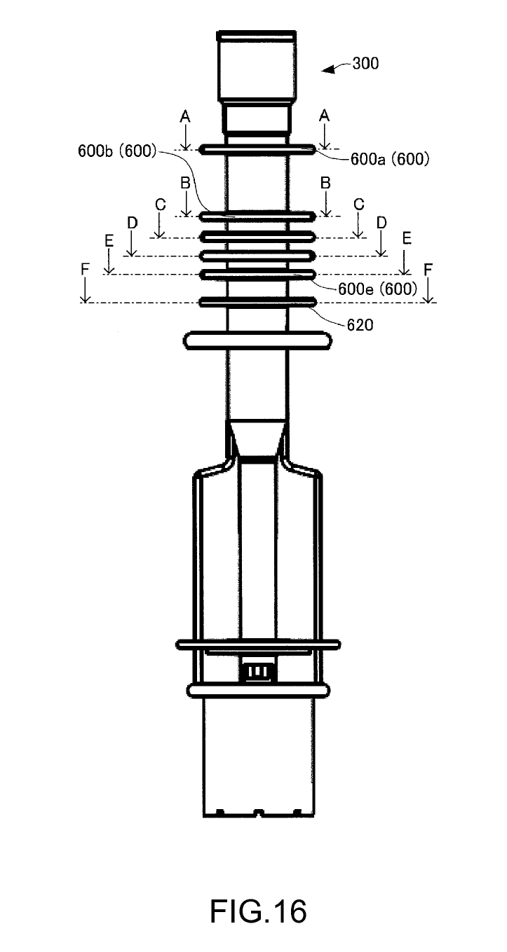

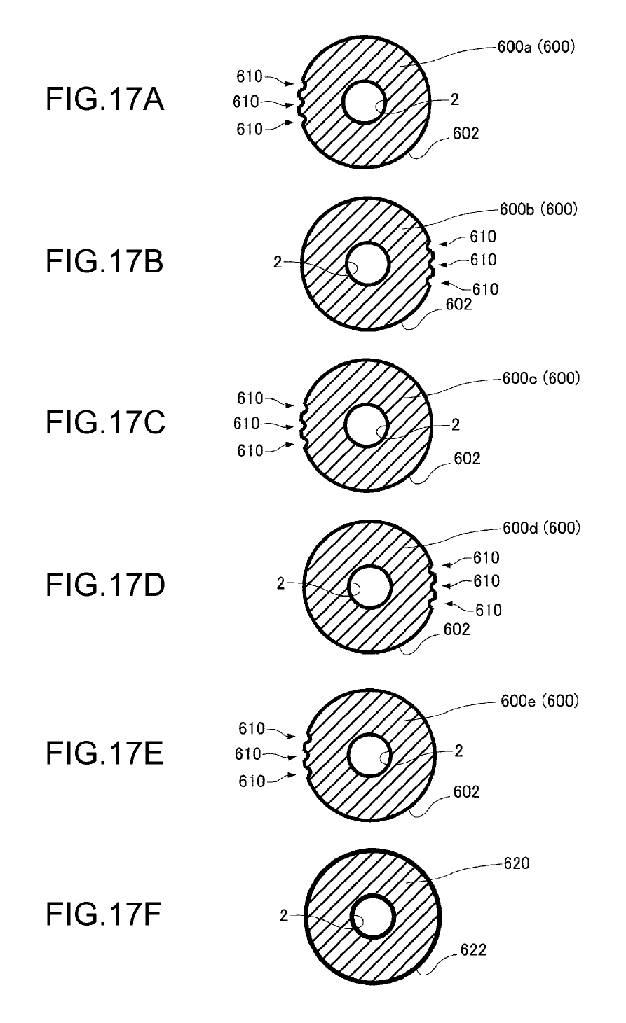

The nucleic acid purification device 5 according to the present embodiment will be described with reference to FIGS. 11 to 17. FIG. 11 is a perspective view illustrating the third washing container 230. FIG. 12 is a longitudinal sectional view illustrating the third washing container 230. FIG. 13 is a longitudinal sectional view illustrating the elution container 300. FIG. 14 is a longitudinal sectional view illustrating the third washing container 230 and the elution container 300. FIG. 15 is a perspective view illustrating the elution container 300. FIG. 16 is a front view illustrating the elution container 300. FIGS. 17A to 17F are sectional views of the elution container 300.

In addition, FIGS. 11 and 12 illustrate the third washing container 230 before constituting the nucleic acid purification device 5 (in a state before the third washing container 230 is bonded to the second washing container 220 and the elution container 300). FIG. 13 illustrates the elution container 300 before constituting the nucleic acid purification device 5 (in a state before the elution container 300 is bonded to the third washing container 230 and the reaction container 400). FIG. 14 illustrates the state in which the third washing container 230 is bonded to the elution container 300. Further, the contents such as the washing solution and the like are not illustrated in FIG. 14.

Further, FIG. 17A is a sectional view taken along the line A-A in FIG. 16, FIG. 17B is a sectional view taken along the line B-B in FIG. 16, FIG. 17C is a sectional view taken along the line C-C in FIG. 16, FIG. 17D is a sectional view taken along the line D-D in FIG. 16, FIG. 17E is a sectional view taken along the line E-E in FIG. 16, and FIG. 17F is a sectional view taken along the line F-F in FIG. 16.

The nucleic acid purification device 5 includes the washing container 200 and the elution container 300 as illustrated in FIGS. 11 to 17. Here, one third washing container 230 which is a minimum configuration unit as a washing container will be described as the washing container.

### 5-1. Washing Container

The washing container before constituting the nucleic acid purification device 5 will be described with reference to FIGS. 11 and 12. In the third washing container (first container) 230 which is a washing container, the third washing solution (first liquid) 16 which is a washing solution and the third and fourth oils 24 and 26 which are fluids that are not mixed with the third washing solution 16 are sealed by and stored in the channel 2 (first channel 2a) of the third washing container 230.

The third washing container 230 includes the third insertion portion 232 in one end portion of a portion forming the channel 2 (first channel 2a) of the third washing container 230 and the third reception portion 234 in another end portion. The channel 2 (first channel 2a) to be formed in the inside of the third washing container 230 penetrates to the third reception portion 234 from the third insertion portion 232. The outer diameter of the channel 2 is formed to be gradually smaller toward the third insertion portion 232 from the third reception portion 234.

The third insertion portion 232 is approximately cylindrical and includes an outer wall 232a whose transverse section is circular.

The third washing container 230 is formed in the periphery of the third insertion portion 232 and includes a third cover portion (outer peripheral wall) 236 to be opened toward the lower portion from the upper portion of the outer wall 232a.

In the third cover portion 236, the upper end is connected to the outer wall 232a of the third insertion portion 232 and the lower end extends beyond the third insertion portion 232. An inner wall 236a of the third cover portion 236 includes an annular stepped portion 236b whose diameter expands toward the lower portion. The stepped portion 236b is positioned in a portion slightly lower than the lower end of the third insertion portion 232 and a film 232c is attached to the surface thereof.

The third reception portion 234 is approximately cylindrical and includes an inner wall 234a whose transverse section is circular. The inner wall 234a includes a tabular stepped portion 234b whose diameter expands toward the upper portion. The stepped portion 234b is positioned in a portion close to the upper end of the third reception portion 234 and a film 234c is attached to the surface thereof. In addition, the film 234c is not illustrated in FIG. 11.

In the third washing container 230, top and bottom openings are sealed by the films 232c and 234c in a state in which the third oil 24, the third washing solution 16, and the fourth oil 26 are stored in the channel 2 in this order from the third reception portion 234 side. The third washing solution 16 is not mixed with the third oil 24 on an interface 16a and the third washing solution 16 is not mixed with the fourth oil 26 on an interface 16b. Therefore, the third oil 24, the third washing solution 16, and the fourth oil 26 which are sealed by and stored in the third washing container 230 hold the third washing solution 16 in the form of a plug.

### 5-2. Elution Container

The elution container before constituting the nucleic acid purification device 5 will be described with reference to FIG. 13. In the elution container (second container) 300, the eluate (second liquid) 32 and the fourth oil 26 which is the fluid that is not mixed with the eluate 32 are sealed by and stored in the channel 2 (second channel 2b) in the elution container 300.

The shape of the elution container 300 is basically the same as that of the third washing container 230.

The elution container 300 includes the elution insertion portion 302 in one end portion of a portion forming the channel 2 (second channel 2b) of the elution container 300 and the elution reception portion 304 in another end portion. The channel 2 to be formed in the inside of the elution container 300 penetrates to the elution reception portion 304 from the elution insertion portion 302. The outer diameter of the channel 2 is formed to be gradually smaller toward the elution insertion portion 302 from the elution reception portion 304.

The elution insertion portion 302 is approximately cylindrical and includes an outer wall 302a whose transverse section is circular.

The elution container 300 is formed in the periphery of the elution insertion portion 302 and includes an elution cover portion 306 to be opened toward the lower portion from the upper portion of the outer wall 302a.

In the elution cover portion 306, the upper end is connected to the outer wall 302a of the elution insertion portion 302 and the lower end extends beyond the elution insertion portion 302. An inner wall 306a of the elution cover portion 306 includes an annular stepped portion 306b whose diameter expands toward the lower portion. The stepped portion 306b is positioned in a portion slightly lower than the lower end of the elution insertion portion 302 and a film 302c is attached to the surface thereof.

The elution reception portion 304 is approximately cylindrical and includes an inner wall 304a whose transverse section is circular. The inner wall 304a includes a tabular stepped portion 304b whose diameter expands toward the upper portion. The stepped portion 304b is positioned in a portion close to the upper end of the elution reception portion 304 and a film 304c is attached to the surface thereof.

In the elution container 300, top and bottom openings are sealed by the films 302c and 304c in a state in which the fourth oil 26, the eluate 32, and the fourth oil 26 are stored in the channel 2 in this order from the elution reception portion 304 side. The eluate 32 and the fourth oil 26 on the upper side are not mixed with each other on an interface 32a and the eluate 32 and the fourth oil 26 on the lower side are not mixed with each other on an interface 32b. Therefore, the fourth oil 26 and the eluate 32 which are sealed by and stored in the elution container 300 hold the eluate 32 in the form of a plug.

The third washing container 230 and the elution container 300 are bonded to each other by the third insertion portion 232 and the elution reception portion 304 breaking through the films 232c and 304c and inserting the third insertion portion 232 into the elution reception portion 304. Therefore, the channel 2 in the third washing container 230 communicates with the channel 2 in the elution container 300 for the first time when the third insertion portion 232 and the elution reception portion 304 break through the films 232c and 304c.

Further, although not illustrated in the figure, films are attached to the first washing container 210 and the second washing container 220 and the washing containers 210, 220, and 230 are bonded to one another by breaking through the films, thereby obtaining the washing container 200. A film is also attached to the adsorption container 100 and the adsorption container 100, the washing container 200, and the elution container 300 are bonded to one another by breaking through the film, thereby obtaining the nucleic acid purification device 5. In addition, a film is also attached to the reaction container 400 and the adsorption container 100, the washing container 200, the elution container 300, and the reaction container 400 are bonded to one another by breaking through the film, thereby obtaining the container assembly 1.

In the nucleic acid purification device 5 (for example, see FIGS. 1 and 2) in which the third washing container 230 (washing container 200) and the elution container 300 are assembled as described above, the washing container 200 that seals and stores the contents and the elution container 300 that seals and stores the contents are bonded to each other and thus the channel 2 for moving a nucleic acid is formed. Consequently, in the nucleic acid purification device 5, it is possible to prevent the eluate 32 from being contaminated by the third washing solution 16 until the washing container 200 and the elution container 300 are bonded to each other. Further, in the nucleic acid purification device 5, since the mixture of the third washing solution 16 with the eluate 32 is prevented by the fourth oil 26 which is not mixed with respective solutions even after the third washing container 230 and the elution container 300 are bonded to each other, it is possible to prevent the eluate 32 from being contaminated by the third washing solution 16 by means of promptly using the container after assembly.

### 5-3. Bonding Structure

The structure in which the third washing container 230 (washing container 200) is boned to the elution container 300 will be described with reference to FIGS. 14 to 17F.

As described above, the third washing container 230 of the washing container 200 includes the third cover portion (outer peripheral wall) 236. The third cover portion 236 is arranged by being spaced apart from the channel 2 (first channel 2a) of the washing container 200 as illustrated in FIG. 14. The third cover portion 236 accommodates a connection portion 250 in which the channel 2 (first channel 2a) of the washing container 200 is connected to the channel 2 (second channel 2b) of the elution container 300. More specifically, the third cover portion 236 accommodates the third insertion portion 232 of the third washing container 230 and the elution reception portion 304 of the elution container 300. In the nucleic acid purification device 5, the third insertion portion 232 is inserted into the elution reception portion 304 (the washing container 200 is inserted into the elution container 300) and the washing container 200 and the elution container 300 are bonded to each other.

The elution container 300 includes a flange 600. The flange 600 is arranged so as to be in contact with the inner wall 236a of the third cover portion 236. The flange 600 is arranged in the periphery of the channel 2 (second channel 2b) of the elution container 300. The flange 600 is arranged in a cylindrical portion 310 of the elution container 300. In the elution container 300, the cylindrical portion 310 is a portion that forms the channel 2 (first channel 2a) of the elution container 300 and is inserted into the inside of the third cover portion 236. The flange 600 is projected toward the outside from the cylindrical portion 310.

The flange 600 is provided with a notched portion 610 as illustrated in FIGS. 15 and 17A to 17F. In the notched portion 610, the flange 600 penetrates in the longitudinal direction of the channel 2 (the longitudinal direction of the container assembly 1). An outer peripheral portion 602 of the flange 600 is in contact with the entire surface of the third cover portion 236 other than the notched portion 610. That is, the outer peripheral portion 602 of the flange 600 includes a portion which is not in contact with the third cover portion 236 because of the notched portion 610. Because of the notched portion 610, a gap is provided between the flange 600 and the third cover portion 236. That is, since the flange 600 includes a gap in a portion between the third cover portion 236 and the flange 600, the flange 600 is in contact with the inner wall 236a of the third cover portion 236. The flange 600 has a shape in which a notch is provided for an annular (ring-shaped) member.

The elution container 300 includes a plurality of flanges 600. In the example of the figure, the elution container 300 includes five flanges 600 (a first flange 600a, a second flange 600b, a third flange 600c, a fourth flange 600d, and a fifth flange 600e). The flanges 600a, 600b, 600c, 600d, and 600e are provided by being arranged in this order in the insertion direction of the washing container 200 (the longitudinal direction of the channel 2, that is, the direction toward the reaction container 400 from the adsorption container 100). In the example of the figure, the distance between the flange 600a and the flange 600b is longer than the distance between other flanges adjacent to each other (for example, the distance between the flange 600b and the flange 600c). Further, the number of flanges 600 is not particularly limited.

The notched portions 610 are provided in each of the plurality of the flanges 600. In the example illustrated in FIGS. 17A to 17F, three notched portions 610 are provided in each of the flanges 600a, 600b, 600c, 600d, and 600e, but the number of the notched portions is not particularly limited. The planar shape (the shape seen from the insertion direction of the washing container 200) of the notched portion 610 is not particularly limited as long as a gap can be formed between the flange 600 and the third cover portion 236 by the notched portion 610.

The notched portion 610 provided in the first flange 600a and the notched portion 610 provided in the second flange 600b are arranged in positions in which the notched portions 610 do not overlap each other when seen from the insertion direction of the washing container 200. That is, a gap (gap formed by the notched portion 610) between the first flange 600a and the third cover portion 236 and a gap between the second flange 600b and the third cover portion 236 are arranged in positions in which the gaps do not overlap each other.

The notched portion 610 provided in the first flange 600a overlaps with, for example, the notched portion 610 provided in the third flange 600c and the notched portion 610 provided in the fifth flange 600d. The notched portion 610 provided in the second flange 600b overlaps with, for example, the notched portion 610 provided in the fourth flange 600d.

The notched portion 610 provided in the first flange 600a and the notched portion 610 provided in the second flange 600b are provided in positions facing each other by interposing the channel 2 of the elution container 300 therebetween when seen from the insertion direction of the washing container 200 as illustrated in FIGS. 17A to 17F. For example, the first flange 600a and the second flange 600b are in a relationship of 2 rotational symmetry when seen from the insertion direction of the washing container 200.

As illustrated in FIG. 14, a plurality of spaces 700 are partitioned by two flanges 600 among the plurality of flanges 600 and the third cover portion 236. One space 700 among the plurality of spaces 700 communicates with another space 700 adjacent to the one space 700 through the notched portion 610 in a state of being divided by one of the two flanges adjacent to each other. Specifically, the first space 700 partitioned by the flange 600a, the flange 600b, the third cover portion 236, and the cylindrical portion 310 communicates with the second space 700 partitioned by the flange 600b, the flange 600c, the third cover portion 236, and the cylindrical portion 310 through the notched portion 610 provided in the second flange 600b. In this manner, the connection portion 350 in which the washing container 200 is connected to the elution container 300 (portion covered by the third cover portion 236) is provided with a plurality of annular spaces 700 communicating with each other by the notched portion 610.

The elution container 300 includes a seal flange 620 in contact with the inner wall 236a of the third cover portion 236. The seal flange 620 is arranged in the periphery of the channel 2 (second channel 2b) of the elution container 300. The plurality of flanges 600 are arranged more on the connection portion 250 side than the seal flange 620. That is, the seal flange 620 is arranged more on the reaction container 400 side than the fifth flange 600e. A notched portion is not provided in the seal flange 620. The seal flange 620 seals the inner wall 236a of the third cover portion 236. The entire surface of an outer peripheral portion 622 of the seal flange 620 is in contact with, for example, the inner wall 236a of the third cover portion 236. The planar shape of the seal flange 620 is annular as illustrated in FIGS. 17A to 17F. Further, for the sake of convenience of illustration, the seal flange 620 is not illustrated in FIG. 15.

As illustrated in FIG. 14, the seal flange 620 partitions the space 710. More specifically, the space 710 is partitioned by the fifth flange 600e, the seal flange 620, the third cover portion 236, and the cylindrical portion 310. The space 710 communicates with the space 700 partitioned by the flange 600d, the flange 600e, the third cover portion 236, and the cylindrical portion 310.

Moreover, in the description above, the example in which the spaces 710 adjacent to each other communicate with each other due to the notched portion 610 being provided in the outer peripheral portion 602 of the flange 600 has been described, but the spaces 710 adjacent to each other may communicate with each other due to a through hole (not illustrated) provided in the flange 600 because the notched portion 610 is not provided in the outer peripheral portion 602 of the flange 600. Further, the spaces 710 adjacent to each other may communicate with each other due to a groove (not illustrated) provided in the inner wall 236a of the third cover portion 236. In this manner, when the spaces 710 adjacent to each other communicate with each other, the shape of the flange 600 and the shape of the third cover portion 236 are not particularly limited.

According to the nucleic acid purification device 5, the elution container 300 includes a plurality of flanges 600 in contact with the inner wall 236a of the third cover portion (outer peripheral wall) 236 and one space 700 which is partitioned by two flanges 600 adjacent to each other among the plurality of flanges 600 and the third cover portion 236 communicates with another space 700 adjacent to the one space 700 in a state of being divided by one of the two flanges adjacent to each other. Accordingly, in the nucleic acid purification device 5, leakage of some of the fourth oil 26 in the washing container 200 and the fourth oil 26 in the elution container 300 to the outside of the nucleic acid purification device 5 can be prevented while the air (atmosphere) in the third cover portion 236 escapes to the outside when the washing container 200 and the elution container 300 are bonded to each other (when the third insertion portion 232 of the washing container 200 is inserted into the elution reception portion 304 of the elution container 300). More specifically, in the nucleic acid purification device 5, when the spaces 700 adjacent to each other communicate with each other, for example, the air in the third cover portion 236 can escape to the outside. In this manner, an insertion load at the time when the washing container 200 is inserted into the elution container 300 can be reduced. Further, the washing container 200 and the elution container 300 can be bonded to each other before some of the fourth oil 26 in the elution container 300 or the like reaches to the outside of the nucleic acid purification device 5 along the outer wall of the elution container 300 by the plurality of flanges 600. That is, the passage in which some of the fourth oil 26 in the elution container 300 or the like reaches the seal flange 620 along the outer wall of the elution container 300 can be lengthened by the plurality of flanges 600. In this manner, the leakage of the fourth oil 26 to the outside can be prevented.

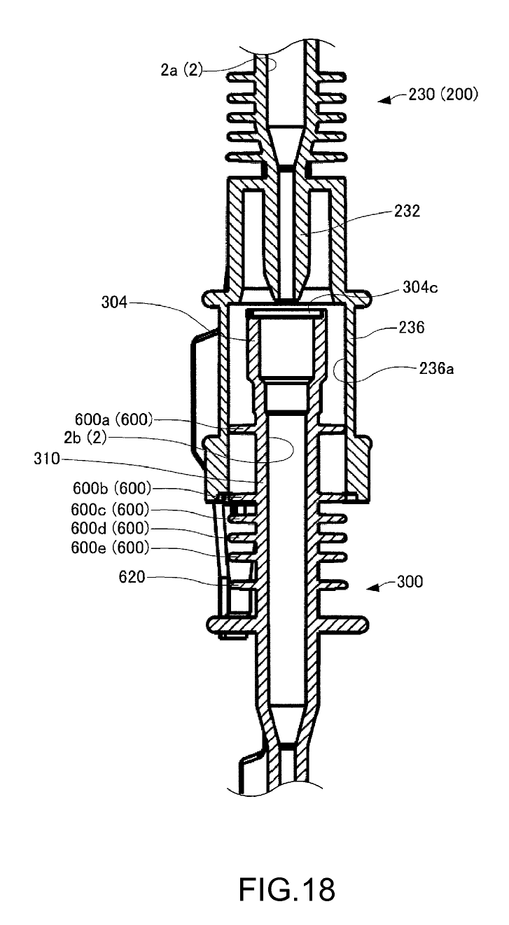

Further, according to the nucleic acid purification device 5, as illustrated in FIG. 18, since the washing container 200 is inserted into the elution container 300 while being in contact with the plurality of flanges 600 when the washing container 200 and the elution container 300 are bonded to each other, the washing container 200 can be stably inserted into the elution container 300. That is, the plurality of flanges 600 can have a function as a guide for inserting the washing container 200 into the elution container 300. Moreover, FIG. 18 is a view illustrating the vertical section of the third washing container 230 and the elution container 300 when the third washing container 230 and the elution container 300 are bonded to each other.

According to the nucleic acid purification device 5, the elution container 300 includes the seal flange 620 in contact with the inner wall 236a of the third cover portion 236, the plurality of flanges 600 are arranged more on the connection portion 250 side than the seal flange 620, and the seal flange 620 seals the inner wall 236a of the third cover portion 236. For this reason, in the nucleic acid purification device 5, the leakage of some of the fourth oil 26 in the elution container 300 or the like to the outside of the nucleic acid purification device 5 can be more reliably prevented by the seal flange 620 when the washing container 200 and the elution container 300 are bonded to each other.

According to the nucleic acid purification device 5, the notched portion 610 is provided in the plurality of flanges 600 and one space 700 communicates with another space 700 through the notched portion 610. For this reason, in the nucleic acid purification device 5, the air in the third cover portion 236 or the like can escape to the outside of the nucleic acid purification device 5 through the notched portion 610 when the washing container 200 and the elution container 300 are bonded to each other. Further, some of the fourth oil 26 in the elution container 300 or the like can be reliably moved from one space 700 to another space 700 (the space 700 positioned below the one space 700) because of a capillary phenomenon in the notched portion 610 when the washing container 200 and the elution container 300 are bonded to each other.

According to the nucleic acid purification device 5, the outer peripheral portion 602 of the plurality of flanges 600 is in contact with the inner wall 236a of the third cover portion 236, excluding the notched portion 610. Accordingly, in the nucleic acid purification device 5, the plurality of flanges 600 can more reliably function as a guide for inserting the washing container 200 to the elution container 300.

According to the nucleic acid purification device 5, the notched portion 610 provided in the first flange 600a and the notched portion 610 provided in the second flange 600b are arranged in positions in which the notched portions do not overlap each other when seen from the insertion direction of the washing container 200. More specifically, the notched portion 610 provided in the first flange 600a and the notched portion 610 provided in the second flange 600b are provided in positions facing each other by interposing the channel 2 of the elution container therebetween. Therefore, in the nucleic acid purification device 5, the passage in which some of the fourth oil 26 in the elution container 300 or the like reaches the seal flange 620 along the outer wall of the elution container 300 can be lengthened by the plurality of flanges 600 when the washing container 200 and the elution container 300 are bonded to each other.

According to the nucleic acid purification device 5, the notched portions 610 provided in each of the flanges 600a, 600b, 600c, 600d, and 600e are present in plural. For this reason, in the nucleic acid purification device 5, the air in the third cover portion 236 or the like can more reliably escape to the outside of the nucleic acid purification device 5 through the notched portion 610 when the washing container 200 and the elution container 300 are bonded to each other. For example, when one notched portion 610 provided in the first flange 600a is present, since the notched portion 610 becomes a channel of the fourth oil 26 at the moment when the fourth oil 26 is brought into contact with the notched portion 610, the air cannot escape to the outside of the nucleic acid purification device 5 through the notched portion 610 in some cases.

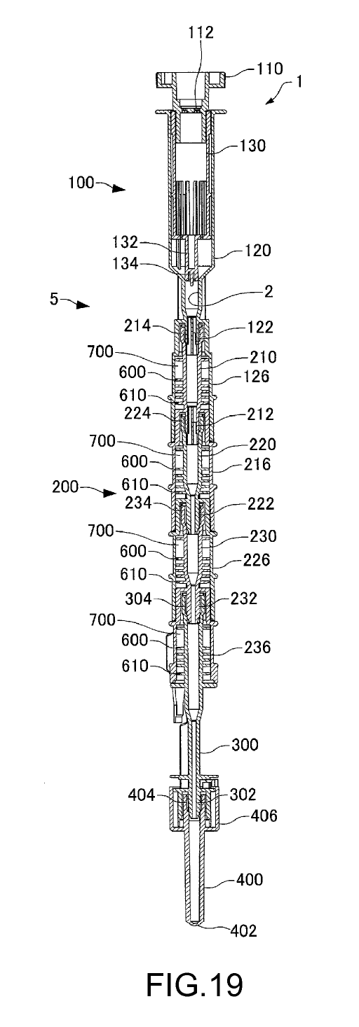

In addition, in the nucleic acid purification device 5, as illustrated in FIG. 19, the washing containers 210, 220, and 230 also include the flange 600 and the seal flange 620 similar to the case of the elution container 300. The flange 600 and the seal flange 620 of the washing containers 210, 220, and 230 have a function which is the same as that of the flange 600 and the seal flange 620 of the elution container 300.

Moreover, the example in which the nucleic acid purification device includes the washing container has been described in the above, but the nucleic acid purification device according to the invention does not include the washing container and the adsorption container may be connected to the elution container in a case where impurities can be removed only by, for example, adsorbing a nucleic acid to the magnetic bead.

The invention is not limited to the above-described embodiments and various modifications are possible. For example, the invention includes configurations (for example, configurations with the same functions, methods, and effects or configurations with the same purposes and effects) which are substantially the same as the configurations described in the embodiments. Further, the invention includes configurations in which parts, which are not indispensable, of the configurations described in the embodiments are replaced. Furthermore, the invention includes configurations exhibiting effects which are the same as those of the configurations described in the embodiments or configurations achieving the purposes which are the same as those of the configurations thereof. Furthermore, the invention includes configurations obtained by applying a known technique to the configurations described in the embodiments.

The entire disclosure of Japanese Patent Application No. 2014-199563, filed Sep. 30, 2014 is expressly incorporated by reference herein.

What is claimed is:

1. A nucleic acid purification device comprising:
a washing container in which a washing solution and a fluid which is not mixed with the washing solution are sealed by and stored in a first channel; and an elution container in which an eluate and a fluid which is not mixed with the eluate are sealed by and stored in a second channel, the washing container and the elution container being bonded to each other to form a channel for moving a nucleic acid,
wherein the washing solution is a liquid which washes a nucleic acid-binding solid phase carrier to which the nucleic acid is adsorbed,
the eluate is a liquid which separates the nucleic acid from the nucleic acid-binding solid phase carrier,
the washing container includes an outer peripheral wall which is arranged by being spaced apart from the first channel and capable of accommodating a connection portion of the first channel and the second channel,
the elution container is arranged in the periphery of the second channel and includes a plurality of flanges in contact with an inner wall of the outer peripheral wall when the washing container and the elution container are bonded to each other,
the plurality of flanges are arranged in a portion which is to be inserted into the inside of the outer peripheral wall of the elution container, and
one space which is partitioned by two flanges adjacent to each other among the plurality of flanges and the outer peripheral wall communicates with another space adjacent to the one space in a state of being divided by one of the two flanges adjacent to each other.

2. The nucleic acid purification device according to claim 1,
wherein the elution container is arranged in the periphery of the second channel and includes a seal flange in contact with the inner wall of the outer peripheral wall,
the plurality of flanges are arranged more on the connection portion side than the seal flange, and
the seal flange seals the inner wall of the outer peripheral wall.

3. The nucleic acid purification device according to claim 1,
wherein the plurality of flanges are provided with a notched portion, and
the one space communicates with the other space by passing through the notched portion.

4. The nucleic acid purification device according to claim 3, wherein an outer peripheral portion of the plurality of flanges is in contact with the inner wall of the outer peripheral wall, excluding the notched portion.

5. A nucleic acid purification device comprising:
a washing container which seals and stores a washing solution and a fluid which is not mixed with the washing solution; and an elution container which seals and stores an eluate and a fluid which is not mixed with the eluate, the washing container and the elution container being bonded to each other to form a channel for moving a nucleic acid,
wherein the washing solution is a liquid which washes a nucleic acid-binding solid phase carrier to which the nucleic acid is adsorbed,
the eluate is a liquid which separates the nucleic acid from the nucleic acid-binding solid phase carrier, and
a connection portion of the washing container and the elution container is provided with a plurality of annular spaces communicating with each other.

6. A nucleic acid purification device comprising:
a first container in which a first liquid and a fluid which is not mixed with the first liquid are sealed by and stored in a first channel; and a second container in which a second liquid and a fluid which is not mixed with the second liquid are sealed by and stored in a second channel, the first container and the second container being bonded to each other to form a channel for moving a nucleic acid,
wherein the first container includes an outer peripheral wall which is arranged in a state of being spaced apart from the first channel and capable of accommodating a connection portion of the first channel and the second channel,
the second container is arranged in the periphery of the second channel and includes a plurality of flanges in contact with an inner wall of the outer peripheral wall when the first container and the second container are bonded to each other,
the plurality of flanges are arranged in a portion which is to be inserted into the inside of the outer peripheral wall of the second container, and
one space which is partitioned by two flanges adjacent to each other among the plurality of flanges and the outer peripheral wall communicates with another space adjacent to the one space in a state of being divided by one of the two flanges adjacent to each other.
